(19) United States
(12) Patent Application Publication
(10) Pub. No.: US 2011/0112394 A1
(43) Pub. Date: May 12, 2011

(54) NEUROMODULATION OF DEEP-BRAIN TARGETS USING FOCUSED ULTRASOUND

(76) Inventor: David J. Mishelevich, Playa del Ray, CA (US)

(21) Appl. No.: 12/940,052

(22) Filed: Nov. 5, 2010

Related U.S. Application Data
(60) Provisional application No. 61/260,172, filed on Nov. 11, 2009.
(60) Provisional application No. 61/295,757, filed on Jan. 17, 2010.

(51) Int. Cl. A61B 5/055; A61N 7/00; A61N 1/05; A61N 2/00; A61N 5/06

(52) U.S. Cl. 600/411; 601/2; 607/45; 600/9; 607/88

(57) ABSTRACT

Disclosed are methods and systems for non-invasive deep brain or superficial neuromodulation for up-regulation or down-regulation using ultrasound impacting one or multiple points in a neural circuit to produce Long-Term Potentiation (LTP) or Long-Term Depression (LTD) to treat indications such as neurologic and psychiatric conditions. Ultrasound transducers are positioned by spinning them around the head on a track, as well as individually rotated or not, with control of direction of the energy emission, intensity, frequency, and phase/intensity relationships to targeting and accomplishing up-regulation and/or down-regulation. Alternatively the ultrasound transducers may be at fixed locations on the track. Use of ancillary monitoring or imaging to provide is optional.

### CROSS REFERENCE TO RELATED APPLICATIONS

This patent application claims priority to provisional patent applications Application No. 61/260,172, filed Nov. 11, 2009, entitled “STIMULATION OF DEEP BRAIN TARGETS USING FOCUSED ULTRASOUND FILED” and Application No. 61/295,757 filed Jan. 17,2010, entitled “NEUROMODULATION OF DEEP BRAIN TARGETS USING FOCUSED ULTRASOUND.” The disclosures of each of these patent applications are herein incorporated by reference in their entirety.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

Described herein are systems and methods for Ultrasound Neuromodulation including one or more ultrasound sources for neuromodulation of target deep brain regions to up-regulate or down-regulate neural activity.

### BACKGROUND OF THE INVENTION

It has been demonstrated that focused ultrasound directed at neural structures can stimulate those structures. If neural activity is increased or excited, the neural structure is said to be up-regulated; if neural activated is decreased or inhibited, the neural structure is said to be down-regulated. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996;22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels which resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to effect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 300 Hz.) are inhibitory. High frequencies (defined as being in the range of 500 Hz to 5 MHz are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

An alternative approach is described by Deisseroth and Schneider (U.S. patent application Ser. No. 12/263,026 published as US 2009/0112133 A1, Apr. 30, 2009) in which modification of neural transmission patterns between neural structures and/or regions is described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February;24(2):275-83 and Clement G T, Hynynen K (2002) A non-invasive method for focusing ultrasound through the human skull. Phys Med Biol 47: 1219-1236.) . Ultrasound can be focused to 0.5 to 2 mm as TMS to 1 cm at best.

### SUMMARY OF THE INVENTION

It is the purpose of this invention to provide methods and systems for non-invasive deep brain or superficial neuromodulation using ultrasound impacting one or multiple points in a neural circuit to produce acute effects on Long-Term Potentiation (LTP) or Long-Term Depression (LTD). Sonic transducers are positioned by spinning them around the head on a track with under control of direction of the energy emission, control of intensity for up-regulation or down-regulation, and control of frequency and phase for focusing on neural targets. The transducer may also rotate while it is moving around the track to enhance ultrasound targeting and delivery. Alternatively the ultrasound transducers may be fixed to the track. Use of ancillary monitoring or imaging to provide feedback is optional. In embodiments were concurrent imaging is to be done, the device of the invention is to be constructed of non-ferrous material. The apparatus can also be optionally covered by a shell.

The targeting can be done with one or more of known external landmarks, an atlas-based approach (e.g., Tailarach or other atlas used in neurosurgery) or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target. For example, some targets, like the Cingulate Gyrus, are elongated and will be more effectively served with an elongated ultrasound field at the target.

### BRIEF DESCRIPTION OF THE DRAWINGS

FIG. 1 shows top and frontal views of the track around the head on which transducers run.

FIG. 2 illustrates the frontal and side views of an example of the transducer with its hemispheric ultrasound array.

FIG. 3 shows an alternative embodiment in which the transducer is rotated while it is going around the track.

FIG. 4 illustrates an embodiment in which the apparatus is enclosed within a shell.

FIG. 5 shows a block diagram of the control circuit.

FIG. 6 illustrates a simplified neural circuit for addiction.

FIG. 7 illustrates targeting multiple targets in a neural circuit for addiction.

FIG. 8 demonstrates using a patient-specific holder to fix the transducers relative to the target.

FIG. 9 shows an embodiment where the transducers can be moved in and out for patient-specific targeting.

### DETAILED DESCRIPTION OF THE INVENTION

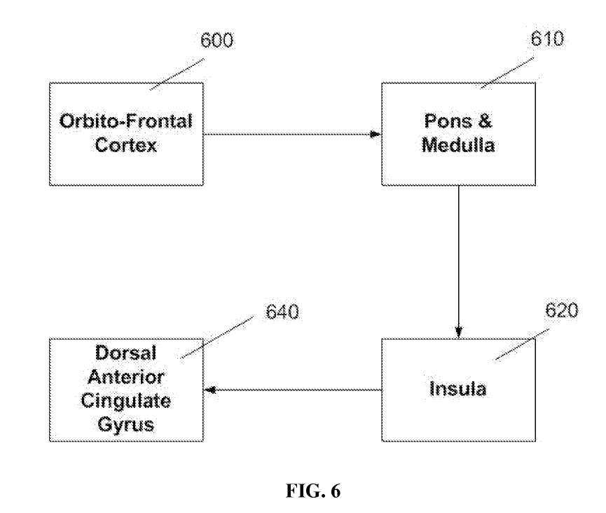

It is the purpose of this invention to provide methods and systems and methods for deep brain or superficial neuromodulation using ultrasound impacting one or multiple points in a neural circuit to produce acute effects or Long-Term Potentiation (LTP) or Long-Term Depression (LTD). For example, FIG. 6 illustrates the neural circuit for addiction.

The stimulation frequency for inhibition is 300 Hz or lower (depending on condition and patient). The stimulation frequency for excitation is in the range of 500 Hz to 5MHz. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down-regulated to achieve the desired effect, it may be preferable to up-regulate its reciprocal more-easily-accessed or safer reciprocal target instead. The frequency range allows penetration through the skull balanced with good neural-tissue absorption. In other embodiments, ultrasound therapy is combined with therapy using other neuromodulation devices (e.g., Transcranial Magnetic Stimulation (TMS), transcranial Direct Current Stimulation (tDCS), and/or Deep Brain Stimulation (DBS) using implanted electrodes). In other embodiments, ultrasound therapy is replaced with one or more therapies selected from one or more modalities of Radio-Frequency (RF) therapy, Transcranial Magnetic Stimulation (TMS), transcranial Direct Current Stimulation (tDCS), or Deep Brain Stimulation (DBS) using implanted electrodes.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 MHz, the value would be 2 mm Thus in the range of 0.4 MHz to 0.7 MHz, for this transducer, the spot sizes will be on the order of 5 mm at the low frequency and 2.8 mm at the high frequency.

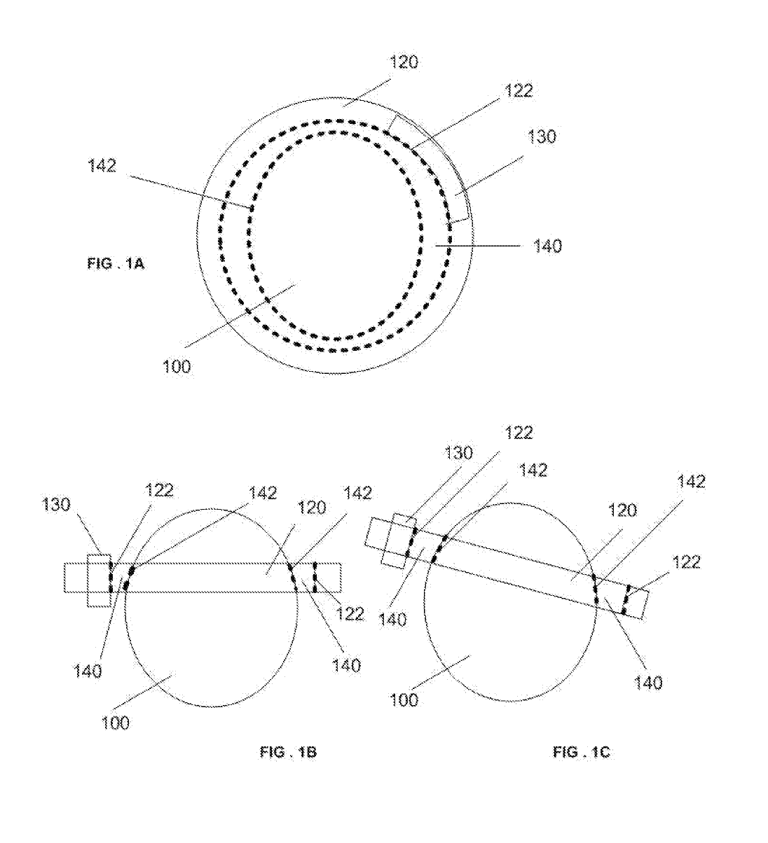

FIG. 1A shows the top view of one embodiment in which a track 120 surrounding human or animal head 100. Riding around track 120 is ultrasound transducer 130. In this embodiment, the face of transducer 130 always faces head 100. Track 120 includes rails for electrical connections to the ultrasound transducers 130. Transducer 130 can ride above the track 120, on the inside of the track 120, or below the track 120. In the latter case, the patient would have less of the apparatus covering their face. In some embodiments, more than one transducer 130 can ride on track 120. For the ultrasound to be effectively transmitted to and through the skull and to brain targets, coupling must be put into place. Ultrasound transmission medium (e.g., silicone oil in a containment pouch) 140 is interposed with one mechanical interface to the ultrasound transducer 130 (completed by a layer of ultrasound transmission gel 122) and the other mechanical interface to the head 100 (completed by a layer of ultrasound transmission gel 142). FIG. 1B shows the frontal view FIG. 1A for the case where transducer 130 is riding on the inside of track 120. The sound-conduction path between ultrasound transducer 130 and head 100 by conductive-gel layer 122, sound-conduction medium 140 and conductive-gel layer 142. FIG. 1C illustrates the situation where track 120 is tilted to allow better positioning for some targets or sets of targets if more than one neural structure is targeted in a given configuration. Again, ultrasound transmission medium 140 is interposed with one mechanical interface to the ultrasound transducer 130 (completed by a layer of ultrasound transmission gel 122) and the other mechanical interface to the head 100 (completed by a layer of ultrasound transmission gel 142). The depth of the point where the ultrasound is focused depends on the shape of the transducer and setting of the phase and amplitude relationships of the elements of the ultrasound transducer array discussed in relation to FIG. 2. In another embodiment, a non-beam-steered-array ultrasound transducer as discussed in relation to FIG. 10 can be used with the transducer only activated when it is correctly positioned to effectively aim at the target. As noted previously, in any case, the ultrasound transducer must be coupled to the head by an ultrasound transmission medium, including gel, if appropriate for effective ultrasound transmission can occur.

In another embodiment of the configuration shown in FIG. 1, instead of the transducer or transducers 130 riding around on the track 120, they may fixed in place at a given location or locations on the track suitable to hit the desired target(s). In this case, in an alternative embodiment, a non-beam-steered-array ultrasound transducer as discussed in relation to FIG. 10 can be used. Again, ultrasound transmission medium must be used for energy coupling.

FIG. 2 shows the face of transducer 230 with an array of ultrasound transducers distributed over the face of transducer array assembly 210. FIG. 2A shows the front of the transducer as would face the target and FIG. 2B shows a side view. Transducer array assemblies of this type may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Keramos-Etalon in the U.S. is another custom-transducer supplier and Blatek is another. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations. FIG. 2C illustrates the ultrasound field represented by dashed lines 240 striking target neural structure 230 with the control of phase and amplitude producing the focus.

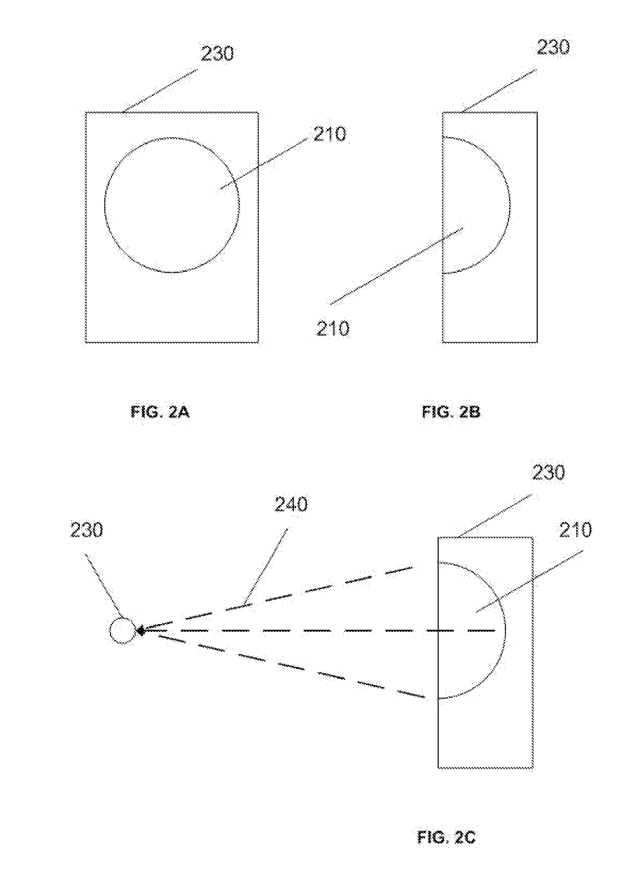

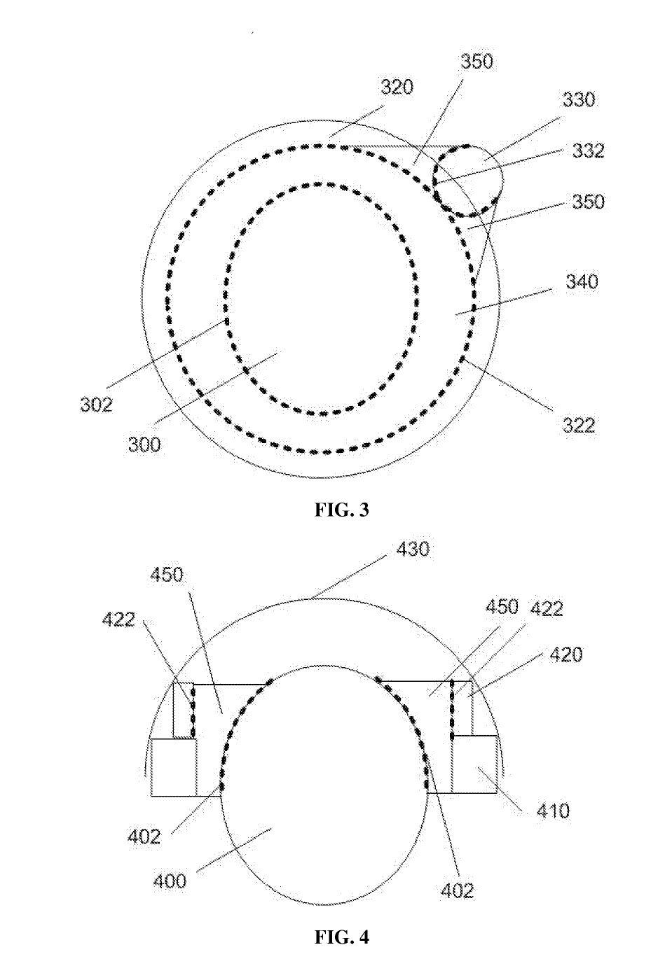

FIG. 3 illustrates an alternative embodiment where track 320 surrounds head 300 now has a transducer 330 whose face can be rotated so it can be aimed towards the intended target(s) rather than always facing perpendicularly to the head. Track 320 includes rails for electrical connections to the sound transducers 330. As transducer 330 reaches a given point on track 300, transducer 330 can be rotated toward the target(s). Again, in some embodiments, more than one transducer 330 can ride on track 320. For the ultrasound to be effectively transmitted to and through the skull and to brain targets, coupling must be put into place. Ultrasound transmission medium 340 is interposed with one mechanical interface to the ultrasound transducer 332 (completed by a layer of ultrasound transmission gel 322) and the other mechanical interface to the head 300 (completed by a layer of ultrasound transmission gel 302). For the rotating element 330, completion of the coupling is achieved with transmission coupling medium 350 is in place (completed by a layer of ultrasound transmission gel 322). In another embodiment, one or more transducers 330 can be fixed in position on track 320, but one or more of transducers 330 can still be rotated to it can be aimed towards the target. Such rotation can either allow sweeping over an elongated target or can periodically alternatively aimed toward each of more than one target. In some embodiments, one or more transducers fixed in position on the track are not rotated. The transducer arrays incorporated in transducer 130 in FIGS. 1 and 330 in FIG. 3 can both of the form of FIG. 2 or other suitable configuration. In addition the tracks in the configurations shown in FIG. 1, FIG. 3 and their alternative embodiments can be raised and lowered vertically as required for optimal targeting. The track can be tilted side to side, front to back, diagonal, or in any direction according to the targeting need. The tracks can be tilted back and forth according to the targeting need. Also there may be transducer carriers containing a plurality of transducers so the combination can target more than one target simultaneously. Other embodiments may be smaller versions covering only a portion of the skull with the ability to target fewer (simultaneously) or perhaps only one target that can be used both in an increased number of clinical settings or at home. Another embodiment incorporates a transducer-holding device, which is not a track, which holds the ultrasound transducers in fixed positions relative to the target or targets. The locations and orientations of the holders can be calculated by locating the applicable targets relative to atlases of brain structure such as the Tailarach atlas. As noted above, in each case, transmission coupling medium must be in place.

In another embodiment, either of the implementations in FIG. 1 or FIG. 3 can be enclosed in a shell as shown in FIG. 4 where head 400 is shown in a frontal view with transducer 420 riding on track 410 all enclosed in shell 430. In this embodiment, there are two transducers 420, placed 180 degrees apart. In this case, as for the other configurations, for the effective ultrasound transmission to and through the skull and to brain targets, coupling must be put into place. Ultrasound transmission medium 450 is interposed with one mechanical interface to the ultrasound transducer 420 (completed by a layer of ultrasound transmission gel 422) and the other mechanical interface to the head 400 (completed by a layer of ultrasound transmission gel 402). In another embodiment, mechanical perturbations are applied radially or axially to move the ultrasound transducers. This is applicable to a variety of transducer configurations.

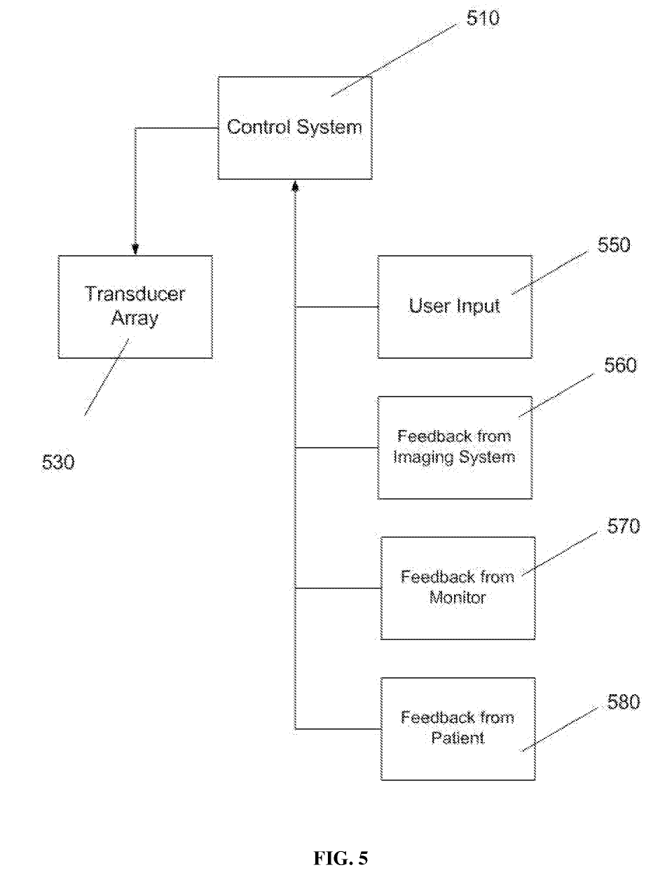

FIG. 5 shows an embodiment of a control circuit. The positioning and emission characteristics of transducer array 530 are controlled by control system 510 with control input from either user by user input 550 and/or from feedback from imaging system 560 (either automatically or display to the user with actual control through user input 550) and/or feedback from a monitor (sound and/or thermal) 570, and/or the patient 580. Control can be provided, as applicable, for direction of the energy emission, intensity, frequency for up-regulation or down-regulation, firing patterns, and phase/intensity relationships for beam steering and focusing on neural targets.

The invention can be applied to a number of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. In addition it can be applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity which can have benefits, for example, in the early treatment of head trauma or other insults to the brain. An example of a neural circuit for a condition, in this case addiction is shown in FIG. 6. In this circuit, the elements are Orbito-Frontal Cortex (OFC) 600, Pons & Medulla 610, Insula 620, and Dorsal Anterior Cingulate Gyms (DACG) 640. One or more targets can be targeted simultaneously or sequentially. Down regulation means that the firing rate of the neural target has its firing rate decreased and thus is inhibited and up regulation means that the firing rate of the neural target has its firing rate increased and thus is excited. For the treatment of addiction, the OFC 600, Insula 620, and DACG 640 would all be down regulated. The ultrasonic firing/timing patterns can be tailored to the response type of a target or the various targets hit within a given neural circuit.

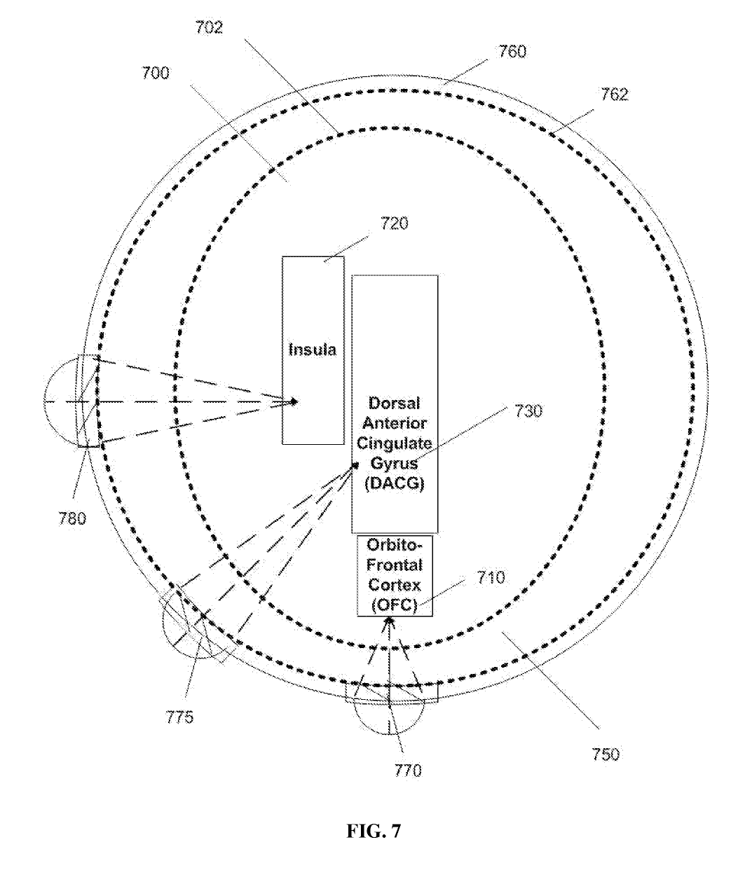

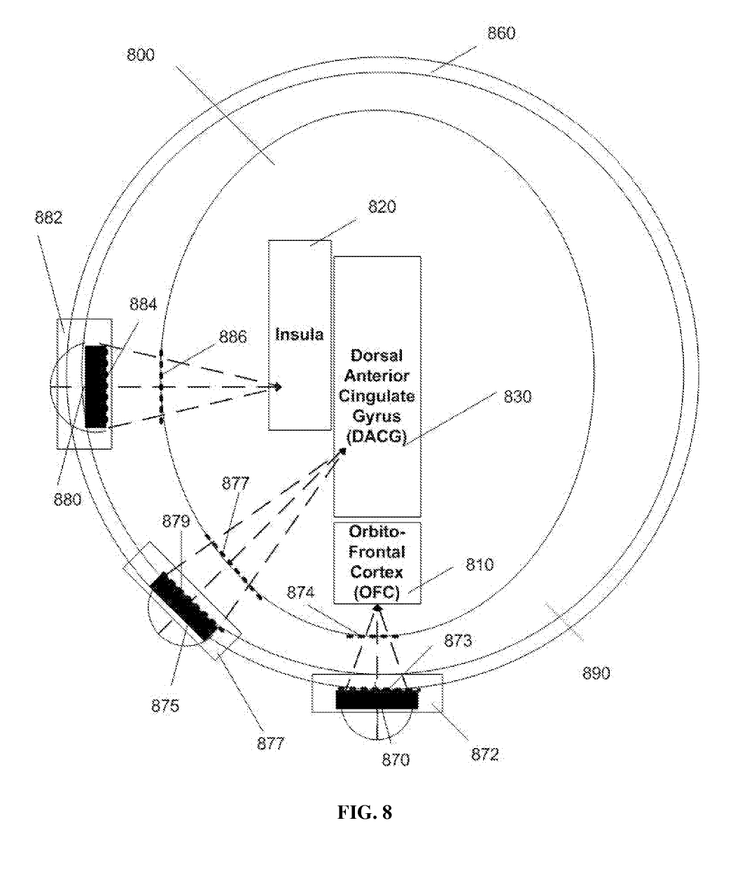

All of the embodiments above, except those explicitly restricted in configuration to hit a single target, are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD) and enhances acute effects as well. In addition, this approach can decrease the number of treatment sessions required for a demonstrated effect and to sustain a long-term effect. Follow-up tune-up sessions at one or more later times may be required. FIG. 7 shows a multi-target configuration. The head 700 contains the three targets, Orbito-Frontal Cortex (OFC) 710, Insula 720, and Dorsal Anterior Cingulate Gyms (DACG) 730, also shown in FIG. 6. These targets are hit by ultrasound transducers 770, 775, and 780, running around track 760 or fixed to track 760. Ultrasound transducer 770 is shown targeting the OFC, transducer 775 is shown targeting the DACG, and transducer 780 is shown targeting the Insula. For the ultrasound to be effectively transmitted to and through the skull and to brain targets, coupling must be put into place. Ultrasound transmission medium 750 is interposed with one mechanical interface to the ultrasound transducers 770, 775, 780 (completed by a layer of ultrasound transmission gel 762) and the other mechanical interface to the head 700 (completed by a layer of ultrasound transmission gel 702). In some cases, the neural structures will be targeted bilaterally (e.g., both the right and the left Insula) and in some cases only one will targeted (e.g., the right Insula in the case of addiction).

FIG. 8 shows a fixed configuration where the appropriate radial (in-out) positions have determined through patient-specific imaging (e.g., PET or fMRI) and the holders positioning the ultrasound transducers are fixed in the determined positions. The head 800 contains the three targets, Orbito-Frontal Cortex (OFC) 810, Insula 820, and Dorsal Anterior Cingulate Gyms (DACG) 830. These targets are hit by ultrasound transducers 870, 875, and 880, fixed to track 860. Ultrasound transducer 870 is shown targeting the OFC, transducer 875 is shown targeting the DACG, and transducer 880 is shown targeting the Insula. Transducer 870 is moved radially in or out of holder 872 and fixed into position. In like manner, transducer 875 is moved radially in or out of holder 877 and fixed into position and transducer 880 is moved radially in or out of holder 882 and fixed into position. For ultrasound to be effectively transmitted to and through the skull and to brain targets, coupling must be put into place. Ultrasound transmission medium 890 is interposed with one mechanical interface to the ultrasound transducers 870, 875, 880 (completed by a layers of ultrasound transmission gel 873, 879, 884) and the other mechanical interface to the head 800 (completed by a layers of ultrasound transmission gel 874, 877, 886). To support this embodiment, treatment planning software is used taking the image-determined target positions and output instructions for manual or computer-aided manufacture of the holders. Alternatively positioning instructions can be output for the operator to position the blocks holding the transducers to be correctly placed relative to the support track. In one embodiment, the transducers positioned using this methodology can be aimed up or down and/or left or right for correct flexible targeting.

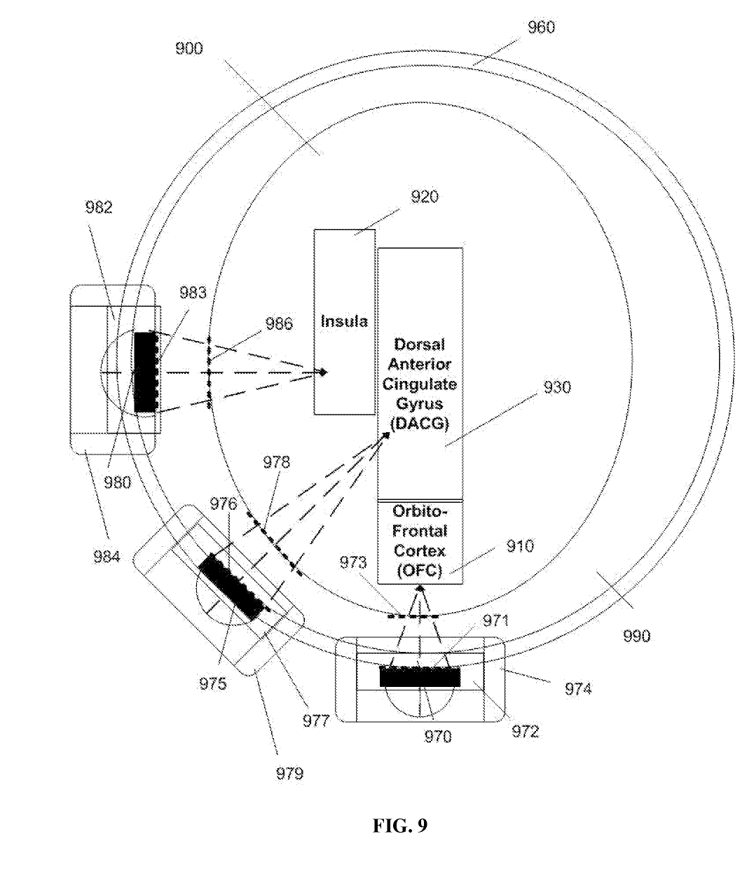

FIG. 9 illustrates an automatically adjustable configuration where based on the image-determined target positions discussed relative to FIG. 8, the transducer holders are moved in or out to the correct positions for the given target without a fixed patient-specific holder having been fabricated or manually adjusted relative to the track or other frame. The head 900 contains the three targets, Orbito-Frontal Cortex (OFC) 910, Insula 920, and Dorsal Anterior Cingulate Gyms (DACG) 930, also shown in FIG. 6. These targets are hit by ultrasound transducers 970, 975, and 980, fixed to track 960. Transducer 970 mounted on support 972 is moved radially in or out of holder 974 by a motor (not shown) to the correct position under control of treatment planning software or manual control. In like manner, transducer 975 mounted on support 977 is moved radially in or out of holder 979 by a motor (not shown) to the correct position under control of treatment planning software or manual control. In like manner, transducer 980 mounted on support 982 is moved radially in or out of holder 984 by a motor (not shown) to the correct position under control of the treatment planning software or manual control. Ultrasound transducer 970 is shown targeting the OFC, transducer 975 is shown targeting the DACG, and transducer 980 is shown targeting the Insula. For the ultrasound to be effectively transmitted to and through the skull and to brain targets, coupling must be put into place. Ultrasound transmission medium 990 is interposed with one mechanical interface to the ultrasound transducers 970, 975, 980 (completed by a layers of ultrasound transmission gels 971, 976, 983) and the other mechanical interface to the head 900 (completed by a layers of ultrasound transmission gel 973, 978, and 986). An embodiment involving the latter would use a single or fewer-than-the-number-of-targets transducers to hit multiple targets since the or fewer-than-the-number-of-targets transducers can be moved in and out or rotated left and right and/or up and down to hit the multiple targets.

The invention allows stimulation adjustments in variables such as, but not limited to, intensity, firing pattern, frequency, phase/intensity relationships, dynamic sweeps, and position to be adjusted so that if a target is in two neuronal circuits the transducer or transducers can be adjusted to get the desired effect and avoid side effects. The side effects could occur because for one indication the given target should be up-regulated and for the other down-regulated. An example is where a target or a nearby target would be down-regulated for one indication such as pain, but up-regulated for another indication such as depression. This scenario applies to either the Dorsal Anterior Cingulate Gyms (DACG) or Caudate Nucleus. Even when a common target is neuromodulated, adjustment of stimulation parameters may moderate or eliminate a problem because of differential effects on the target relative to the involved clinical indications.

The invention also contradictory effects in cases where a target is common to both two neural circuits in another way. This is accomplished by treating (either simultaneously or sequentially, as applicable) other neural-structure targets in the neural circuits in which the given target is a member to counterbalance contradictory side effects. This also applies to situations where a tissue volume of neuromodulation encompasses a plurality of targets. Again, an example is where a target or a nearby target would be down-regulated for one indication such as pain, but up-regulated for another indication such as depression. This scenario applies to the Dorsal Anterior Cingulate Gyms (DACG). To counterbalance the down-regulation of the DACG during treatment for pain that negatively impacts the treatment for depression, one would up-regulate the Nucleus Accumbens or Hippocampus which are other targets in the depression neural circuit. A plurality of such applicable targets could be stimulated as well.

Another applicable scenario is the Nucleus Accumbens which is down-regulated to treat addiction, but up-regulated to treat depression. To counteract the down-regulation of the Nucleus Accumbens to treat depression but will negatively impact the treatment of depression which would like the Nucleus Accumbens to be up-regulated, one would up-regulate the Caudate Nucleus as well. Not only can potential positive impacts be negated, one wants to avoid side effects such as treating depression, but also causing pain. These principles of the invention are applicable whether ultrasound is used alone, in combination with other modalities, or with one or more other modalities of treatment without ultrasound. Any modality involved in a given treatment can have its stimulation characteristics adjusted in concert with the other involved modalities to avoid side effects.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method of neuromodulating one or a plurality of deep-brain targets using ultrasound stimulation, the method comprising:
aiming one or a plurality of ultrasound transducers at one or a plurality of deep-brain targets,
applying power to each of the ultrasound transducers via a control circuit thereby neuromodulating the activity of the deep brain target region,
moving one or a plurality of transducers around a track surrounding the mammal's head.

2. The method of claim 1, further comprising identifying a deep-brain target.

3. The method of claim 1, further comprising where neuromodulation of a plurality of targets is selected from the group consisting of up-regulating all neuronal targets, down-regulating all neuronal targets, up-regulating one or a plurality of neuronal targets and down-regulating the other targets.

4. The method of claim 1, wherein the step of aiming comprising orienting the ultrasound transducer and focusing the ultrasound so that it hits the target.

5. The method of claim 1, wherein the acoustic ultrasound frequency is in the range of 0.3 MHz to 0.8 MHz.

6. The method of claim 1, where in the power applied is selected from group consisting of less than 180 mW/cm² and greater than 180 mW/cm² but less than that causing tissue damage.

7. The method of claim 1, wherein a stimulation frequency of 300 Hz or lower is applied for inhibition of neural activity.

8. The method of claim 1, wherein the stimulation frequency is in the range of 500 Hz to 5 MHz for excitation.

9. The method of claim 1, wherein the focus area of the pulsed ultrasound is selected from the group consisting of 0.5 to 500 mm in diameter and 500 to 1500 mm in diameter.

10. The method of claim 1, wherein the number of ultrasound transducers is between 1 and 25.

11. The method of claim 1, wherein a disorder is treated by neuromodulation, wherein the target brain regions are selected from the group consisting of NeoCortex, any of the subregions of the Pre-Frontal Cortex, Orbito-Frontal Cortex (OFC), Cingulate Genu, subregions of the Cingulate Gyrus, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, or any of the tracts between the brain targets.

12. The method of claim 1, wherein the disorder treated is selected from the group consisting of: addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy.

13. The method of claim 1 where the ultrasound is applied for the purpose selected from the group consisting of: cognitive enhancement, hedonic stimulation, enhancement of neural plasticity, improvement in wakefulness, brain mapping, diagnostic applications, and other research functions.

14. The method of claim 1, wherein mechanical perturbations are applied radially or axially to move the ultrasound transducers.

15. The method of claim 1, wherein a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, patient.

16. The method of claim 1, wherein ultrasound therapy is combined with one or more therapies selected from the group consisting of Radio-Frequency (RF) therapy, Transcranial Magnetic Stimulation (TMS), transcranial Direct Current Stimulation (tDCS), Deep Brain Stimulation (DBS) using implanted electrodes.

17. The method of claim 1 in which one or a plurality of ultrasound transducers moving around a track surrounding the mammal's had are rotated as they go around the track to maintain focus for a longer period of time.

18. The method of claim 1 where the position of one or a plurality of ultrasound transducers are mounted on the track surrounding the mammal's head in a fixed position.

19. The method of claim 1 for neuromodulating a plurality of deep-brain targets using ultrasound stimulation where there are contradictory effects relative to clinical indications, the method comprising:
a. identifying other targets in the neural circuits that impact those clinical indications that are not in common, and
b. up-regulating or down-regulating one or a plurality of those targets, whereby the contradictory effects are minimized.

20. The method of claim 1 wherein ultrasound therapy is replaced with one or more therapies selected from the group consisting of Radio-Frequency (RF) therapy, Transcranial Magnetic Stimulation (TMS), transcranial Direct Current Stimulation (tDCS), Deep Brain Stimulation (DBS) using implanted electrodes.
